(19) United States
(12) Patent Application Publication
(10) Pub. No.: US 2004/0039069 A1
(43) Pub. Date: Feb. 26, 2004

(54) Reformer

(76) Inventors: Tatsuhiko Kiuchi, Chiba (JP); Makoto Shimagaki, Chiba (JP); Kunio Hirotani, Chiba (JP); Katsunori Yagoh, Chiba (JP)

Correspondence Address: KNOBBE MARTENS OLSON & BEAR LLP, 2040 MAIN STREET, FOURTEENTH FLOOR, IRVINE, CA, 92614, US

(21) Appl. No.: 10/633,826

(22) Filed: Aug. 4, 2003

(30) Foreign Application Priority Data
Aug. 14, 2002 (JP) 2002-236447

(51) Int. Cl. B01J 8/04; C07C 27/06

(52) U.S. Cl. 518/726; 422/188; 518/703

(57) ABSTRACT

A reformer for obtaining a synthesis gas by partially oxidizing a carbon-containing raw material and then steam-reforming the oxidized raw material, includes: a reactor vessel, an oxidizing agent feed pipe and a carbon-containing raw material feed pipe, wherein the central axis of the oxidizing agent feed pipe and the central axis of the carbon-containing raw material feed pipe intersect with each other downstream of the outlet of the oxidizing agent feed pipe in an oxidizing agent flowing direction and downstream of the outlet of the carbon-containing raw material feed pipe in a carbon-containing raw material flowing direction.

### BACKGROUND OF THE INVENTION

1. Field of the Invention

The present invention relates to a reformer for producing a synthesis gas including hydrogen and carbon monoxide as essential components by partially oxidizing a carbon-containing raw material such as a natural gas, methane, ethane, propane, butane or naphtha by use of air, oxygen-enriched air or oxygen, and then reforming the oxidized raw material with steam.

2. Description of the Related Art

As a method for producing a synthesis gas from a carbon-containing raw material such as a natural gas or naphtha, used for hydrogen, methanol or ammonia production, a method using two types of reactors is well known.

In production of ammonia using a natural gas as a raw material, the carbon-containing raw material and steam are used as raw materials and a first synthesis gas including hydrogen as an essential component is produced by steam reforming reaction and shift reaction in a first reactor. This reactor is a reforming furnace type reactor in which a number of reaction tubes are placed in a reforming furnace and heat is fed from the outsides of the reaction tubes. The first synthesis gas comprises hydrogen as a main component, carbon monoxide, carbon dioxide, methane and steam. For example, the reaction tubes are made of heat resistant-steel, have an internal diameter of about 100 mm and a length of about 15 m and are filled with a nickel-based catalyst.

Reaction conditions include a pressure of 2 to 4 MPa and a temperature of 700 to 900° C.

Then, together with an oxidizing agent, the first synthesis gas is fed into a second reactor shown in FIG. 4, and the target synthesis gas is produced by use of the following partial oxidation reaction. For example, this reactor is filled with the nickel-based catalyst as in the case of the first reactor. Reaction conditions include a pressure of 2 to 4 MPa and a temperature of 900 to 1,1000° C., at the exit of the second reactor.

As the oxidizing agent, air, oxygen or oxygen-enriched air is selected and used in consideration of economical efficiency.

It is considered that reactions occur in two steps in the second reactor. Firstly, in a first step, a high temperature gas is generated by use of the partial oxidation reaction between the first synthesis gas fed from the first reactor and the oxidizing agent. Then, in a second step, the produced partial oxidation reaction gas is fed into a catalyst layer so as to produce the synthesis gas.

To be more specific, in the first step in the second reactor, the partial oxidation reaction, which is an exothermic reaction, occurs. Taking methane which is a component of the first synthesis gas for example, a typical partial oxidation reaction is represented by the following equation:

2CH₄+3O₂→2CO+4H₂O

exothermic reaction

In the second step, in the catalyst layer disposed downstream of the partial oxidation reaction zone, the following steam reforming reaction and shift reaction occur thereby producing the synthesis gas.

CH₄+H₂O [symbol] CO+3H₂

endothermic reaction (hydrogen producing direction)

CO+H₂O [symbol] H₂+CO₂

exothermic reaction (hydrogen producing direction)

In the above two reactions, the steam reforming reaction is predominant. Thus, as a whole, the reactions act as an endothermic reaction in total.

Heat required for the endothermic reaction in the second step is covered by the heat generated from the partial oxidation reaction in the first step. Thus, it is desirable that the high temperature partial oxidation reaction gas produced in the first step be uniformly fed to the catalyst layer for the second step.

The second reactor (hereinafter referred to as “reformer”) which is industrially used is shown in FIG. 4. The reactor is described in “Ammonia Plant Safety & Related Facilities”, Volume 34, pages 24.

As shown in FIG. 4, the reformer comprises a reactor vessel 4, an oxidizing agent feed pipe 21, a carbon-containing raw material feed pipe 11, a burner nozzle 6, bricks 3, a catalyst layer 5, alumina balls 7, and support bricks 8. An oxidizing agent 2 is fed through the cylindrical oxidizing agent feed pipe 21, and a carbon-containing raw material 1 is fed through the carbon-containing raw material feed pipe 11 which is disposed in the lateral or axial direction of the oxidizing agent feed pipe 21 and discharged around the oxidizing agent feed pipe 21. The fed carbon-containing raw material 1 flows downward along the oxidizing agent feed pipe 21 and merges with the oxidizing agent in the vicinity of the burner nozzle so as to cause a partial oxidation reaction, thereby forming a high temperature zone. The high temperature partial oxidation reaction gas 31 is fed to the catalyst layer 5 having the heat resistant bricks 3 disposed thereon so as to further cause a steam reforming reaction and a shift reaction and passes through the alumina balls 7 and the support bricks 8. Thereby, a target synthesis gas 32 is obtained from a synthesis gas outlet 9.

Structures related with the burner nozzle 6 where the high temperature zone is formed are disclosed in Japanese Patent Application Laid-Open Nos. H05-256420 (1993), H06-34112 (1994) and 2000-186806.

Japanese Patent Application Laid-Open No. H05-256420(1993) discloses a structure in which a fuel gas injector is connected to the flow-out end of a fuel gas passage, the fuel gas injector has an internal surface having a U-shaped cross section around an axis common to a burner tube and an ejector, an oxide injector is connected to the flow-out end of an oxide feed pipe, the oxide injector has a surface having a U-shaped cross section coaxially apart from the fuel gas injector, each injection chamber has a U-shaped profile and has a circular flow-out end around the common axis, a tubular protrusion is disposed coaxially in an oxide injection chamber, the protrusion has a dome-shaped upstream end and a tapered downstream end, a swirler is provided for a protrusion between the upstream and downstream ends thereof, and the swirler has static swirling blades extended on the surface of the oxide injection chamber.

Further, Japanese Patent Application Laid-Open No. H06-34112 (1994) discloses a composite lance gas injector in which a composite lance which comprises a metallic feed pipe, a nozzle having a metallic rear piece communicated with the feed pipe and a ceramic front piece adjacent to the rear piece and means for providing a protective gas around the nozzle is disposed in a recessed position in a cavity present in a wall of a combustion zone and having an opening communicated with the combustion zone.

Japanese Patent Application Laid-Open No. 2000-186806 discloses a method for protecting a burner body by flowing a protective atmosphere along the external surface and whole surface of the burner body in an amount sufficient to dilute or replace a corrosive atmosphere around the surface of the burner when a hydrocarbon fuel is combusted by the burner exposed to the corrosive atmosphere.

In these techniques, a part intended for achieving promotion of mixing, uniform mixing, prevention of back-flow, adjustment of injection direction, cooling or the like is attached to or near the tip of the burner nozzle, thereby giving the burner nozzle a complex configuration. Further, in these techniques, since the direction in which the carbon-containing raw material flows until the partial oxidation reaction occurs is parallel to the oxidizing agent feed pipe, the high temperature partial oxidation reaction zone at the outlet of the burner nozzle remarkably lengthens downward.

A burner nozzle having a complex shape is liable to be damaged by heat or have high temperature corrosion due to the high temperature partial oxidation reaction gas in the vicinity of the burner nozzle and can hardly endure long-term use. Further, when a local high temperature partial oxidation reaction zone right under the burner nozzle extends downward, the catalyst may be damaged, the temperature of gas reaching the surface of the catalyst layer may be not uniform, and reaction efficiencies of the steam reforming reaction and shift reaction occurring inside the catalyst layer may be lower.

### SUMMARY OF THE INVENTION

An object of the present invention is to provide a long-life reformer capable of preventing generation of high temperature zone in the vicinity of burner nozzle caused by a partial oxidation reaction even using a burner having a simple structure, causing the partial oxidation reaction to proceed stably and uniformly, and preventing the burner and a catalyst from being damaged by heat or high temperature corrosion. Another object of the present invention is to provide a reformer capable of keeping reaction efficiencies of reactions occurring in a catalyst layer at a high level.

The inventors have found that when an oxidizing agent and a carbon-containing raw material are mixed together in a specific manner, generation of high temperature zone in the vicinity of burner nozzle by a partial oxidation reaction may be prevented, and the partial oxidation reaction may also proceed stably and uniformly. The present invention has been completed based on this finding.

The present invention provides a reformer for obtaining a synthesis gas by partially oxidizing a carbon-containing raw material and then steam reforming the oxidized raw material, the reformer comprising:

a reactor vessel,

an oxidizing agent feed pipe for feeding an oxidizing agent into the vessel, and

a carbon-containing raw material feed pipe for feeding the carbon-containing raw material into the vessel,

wherein the central axis of the oxidizing agent feed pipe and the central axis of the carbon-containing raw material feed pipe intersect with each other in the downstream of the outlet of the oxidizing agent feed pipe in an oxidizing agent flowing direction and in the downstream of the outlet of the carbon-containing raw material feed pipe in a carbon-containing raw material flowing direction.

In this reformer, the central axis of the oxidizing agent feed pipe and the central axis of the carbon-containing raw material feed pipe preferably intersect with each other at an angle of 80 to 100 degree.

Further, the following relationships are preferably satisfied:

40≦V1≦150;

0.2V1≦V2≦0.8V1; and

min(0.5D2, 7.0D1)≦L1≦max(0.5D2, 7.0D1)

wherein D1 (m) is an equivalent hydraulic diameter of the outlet of the oxidizing agent feed pipe,

D2 (m) is an equivalent hydraulic diameter of the outlet of the carbon-containing raw material feed pipe,

V1 (m/sec) is an average flow velocity of oxidizing agent jet at the outlet of the oxidizing agent feed pipe,

V2 (m/sec) is an average flow velocity of carbon-containing raw material jet at the outlet of the carbon-containing raw material feed pipe, and

L1 (m) is a distance from the outlet-end of the oxidizing agent feed pipe to an intersection point where the central axis of the oxidizing agent feed pipe and the central axis of the carbon-containing raw material feed pipe intersect with each other.

Further, the cross section of the outlet of the oxidizing agent feed pipe preferably is circular, oval, polygonal, star-shape or petal-shape.

Min(0.5D2, 7.0D1) is a function giving a minimum value out of 0.5D2 and 7.0D1, and max(0.5D2, 7.0D1) is a function giving a maximum value out of 0.5D2 and 7.0D1. Further, V1 and V2 are flow velocities at actual temperatures and pressures.

### BRIEF DESCRIPTION OF THE DRAWINGS

FIG. 1 is a schematic showing an embodiment of a reformer of the present invention.

FIG. 2 is an enlarged view of the upper part of the reformer of FIG. 1.

FIG. 3 is a schematic showing shapes which may be applied to the cross section of the outlet of an oxidizing agent feed pipe used in the present invention.

FIG. 4 is a schematic showing a reformer of the prior art.

### DETAILED DESCRIPTION OF THE INVENTION

As a catalyst, there can be used any catalyst which is heat resistant and active in a steam reforming reaction and a shift reaction. For example, a commercially available nickel-based catalyst may be used. As for the reaction conditions, the pressure is preferably 2 to 6 MPa, and the temperature is preferably 800 to 1,100° C. The temperature and pressure are selected in view of economical efficiency.

Further, an oxidizing agent is selected from oxygen, air and oxygen-enriched air in accordance with a product to be produced and in view of economical efficiency. For example, in production of synthesis gases for producing methanol or hydrogen, it may be economical that the oxidizing agent contains as little nitrogen as possible. From this viewpoint, it is preferred to use oxygen obtained from a air separation plant. Meanwhile, in production of a synthesis gas for producing ammonia, it is more desirable that the oxidizing agent contains nitrogen which is a raw material for synthesizing ammonia. Hence, it is preferred to use air or oxygen-enriched air. The feed rate of air is determined as appropriate in consideration of economical efficiency of ammonia production. Further, the feed rate and oxygen concentration of oxygen-enriched air are also determined as appropriate in consideration of economical efficiency of ammonia production.

As a carbon-containing raw material, a compound which has one or more carbon atoms and can generate hydrogen by steam reforming reaction may be used alone or in a mixture of two or more. The carbon-containing raw material may contain other components. Illustrative examples of the carbon-containing raw material include a natural gas, methane, ethane, propane, butane and naphtha. Further, the carbon-containing raw material such as a natural gas may also contain hydrogen, carbon monoxide, carbon dioxide and steam. For example, a partially reformed gas resulting from partial reforming of the carbon-containing raw material such as a natural gas may be used as a carbon-containing raw material. From a practical standpoint for preventing carbon deposition, it is preferable that the carbon-containing raw material contains steam, and the steam may be used as a reactant for the steam reforming reaction.

Hereinafter, an embodiment of the present invention will be described with reference to the drawings. However, the present invention shall not be limited to the embodiment.

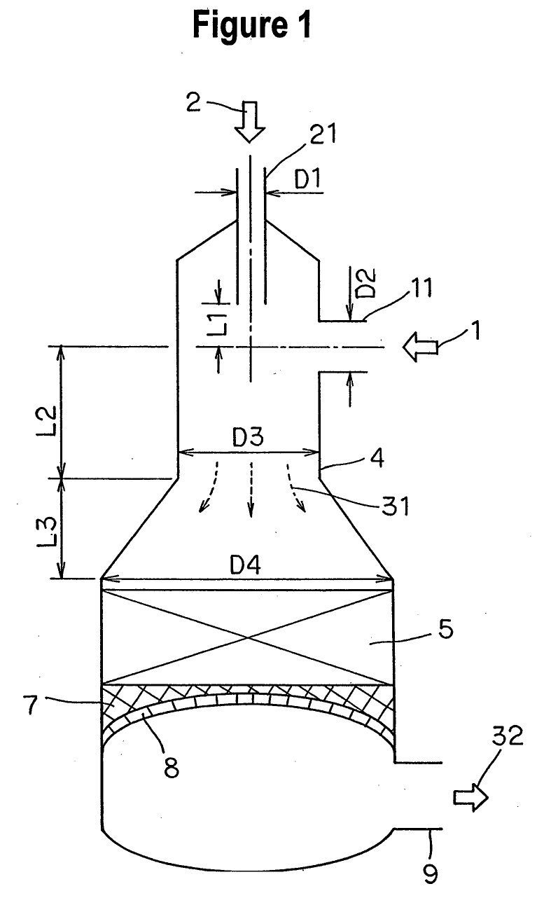

FIG. 1 is a schematic showing the whole of an embodiment of a reformer of the present invention. The reformer comprises a reactor vessel 4 equipped with an oxidizing agent feed pipe 21 for feeding an oxidizing agent 2 into the vessel, a carbon-containing raw material feed pipe 11 for feeding a carbon-containing raw material 1 into the vessel, a region for accommodating a catalyst layer 5 for promoting a steam reforming reaction and a shift reaction, a region for accommodating an alumina ball layer 7 for supporting the catalyst layer and equalizing pressure drops in the reformer, the alumina ball layer being located downstream of the catalyst layer, support bricks 8 for supporting the catalyst layer, the alumina ball layer and the like, and an outlet 9 for discharging a synthesis gas 32. The reactor vessel is closed to the atmosphere.

Here, the oxidizing agent feed pipe and the carbon-containing raw material feed pipe are cylindrical pipes. Further, the reformer has a partial oxidation reaction part (corresponding to a cylindrical part having an internal diameter of D3, and hereinafter may be referred to as “upper part of the reformer”) for mixing an oxidizing agent and a carbon-containing raw material to cause a partial oxidation reaction, the partial oxidation reaction part having the oxidizing agent feed pipe and the carbon-containing raw material feed pipe; a conical part for expanding the flow of a partial oxidation reaction gas 31 which is a partially oxidized gas, the conical part having an internal diameter which continuously expands from D3 to D4; and a steam reforming part (corresponding to a cylindrical portion having an internal diameter of D4, and hereinafter may be referred to as “lower part of the reformer”) for obtaining a synthesis gas from the partial oxidation reaction gas by a steam reforming reaction and a shift reaction, the steam reforming part having a region for accommodating the catalyst layer and the alumina ball layer, the support bricks and the outlet.

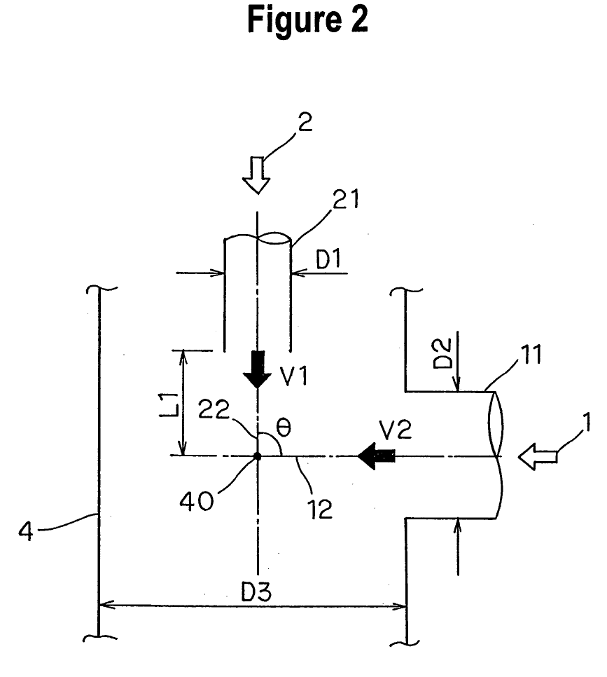

FIG. 2 is an enlarged view of a portion where an oxidizing agent and a carbon-containing fuel are mixed together in the upper part of the reformer. The central axis 22 of the oxidizing agent feed pipe 21 and the central axis 12 of the carbon-containing raw material feed pipe 11 intersect with each other at an intersection point 40. The intersection point 40 is situated on the downstream side of the outlet of the oxidizing agent feed pipe 21 in an oxidizing agent flowing direction and on the downstream side of the outlet of the carbon-containing raw material feed pipe 11 in a carbon-containing raw material flowing direction.

The central axis 22 and the central axis 12 preferably intersect with each other at an angle θ of 80 to 100°, more preferably 90°.

Thus, by intersecting an oxidizing agent jet with a carbon-containing raw material jet, it is possible to effectively use diffusive mixing on surfaces of potential cores formed by these two jets and to accelerate the partial oxidation reaction uniformly. Accordingly, the growth of the oxidizing agent jet can be prevented, and it can also be prevented that a high temperature zone generated by partial oxidation extends downward and focuses near the center of the catalyst layer.

Here, the following relationships are preferably satisfied:

40≦V1≦150,

0.2V1≦V2≦0.8V1, and

min(0.5D2, 7.0D1)≦L1≦max(0.5D2, 7.0D1)

and the following relationships are more preferably satisfied:

60≦V1≦90,

0.4V1≦V2≦0.6V1, and

min(0.6D2, 5.0D1)≦L1≦max(0.6D2, 5.0D1)

wherein D1 (m) is an equivalent hydraulic diameter of the outlet of the oxidizing agent feed pipe,

D2 (m) is an equivalent hydraulic diameter of the outlet of the carbon-containing raw material feed pipe,

V1 (m/sec) is an average flow velocity of oxidizing agent jet at the outlet of the oxidizing agent feed pipe,

V2 (m/sec) is an average flow velocity of carbon-containing raw material jet at the outlet of the carbon-containing raw material feed pipe, and

L1 (m) is a distance from the end of the outlet of the oxidizing agent feed pipe to an intersection point where the central axis of the oxidizing agent feed pipe and the central axis of the carbon-containing raw material feed pipe intersect with each other.

From the viewpoints of preventing the potential core of the oxidizing agent jet from growing and getting longer, and preventing the high temperature zone generated by the partial oxidation reaction from growing in long and narrow shape downward in the oxidizing agent jet heading direction so as to prevent a high temperature gas from converging near the center of the catalyst layer, and preventing the catalyst from being melt-damaged, the average flow velocity V1 of the oxidizing agent is preferably not higher than 150 m/sec, more preferably not higher than 90 m/sec.

From the viewpoints of preventing the high temperature partial oxidation reaction zone from converging near the outlet of the oxidizing agent feed pipe so as to prevent the oxidizing agent feed pipe from being melt-damaged and preventing the high temperature partial oxidation reaction zone from inclining to a reformer furnace wall opposite to the carbon-containing raw material feed pipe so as to prevent the furnace wall from being damaged, the average flow velocity V1 of the oxidizing agent is preferably not lower than 40 m/sec, more preferably not lower than 60 m/sec. Thus, by avoiding formation of the high temperature partial oxidation reaction zone near the tip of the oxidizing agent feed pipe, it is possible to obviate the need for providing a water-cooling mechanism at the tip of the nozzle so as to prevent the nozzle for being damaged by heat.

From the viewpoint of preventing the high temperature gas from flowing excessively on the reformer furnace wall opposite to the carbon-containing raw material feed pipe so as to prevent the furnace wall from being damaged, the average flow velocity V2 of the carbon-containing raw material is preferably not higher than 0.8V1, more preferably not higher than 0.6V1.

From the viewpoint of preventing the potential core of the oxidizing agent jet from growing and lengthening so as to prevent the high temperature gas from converging near the center of the catalyst layer and prevent the catalyst from being melt-damaged, the average flow velocity V2 of the carbon-containing raw material is preferably not lower than 0.2V1, more preferably not lower than 0.4V1.

L1 is preferably not smaller than 0.5D2 (when 0.5D2≦7.0D1) or not smaller than 7.0D1 (when 0.5D2>7.0D1), more preferably not smaller than 0.6D2 (when 0.6D2≦5.0D1) or not smaller than 5.0D1 (when 0.6D2>5.0D1).

L1 is preferably not larger than 7.0D1 (when 0.5D2≦7.0D1) or not larger than 0.5D2 (when 0.5D2>7.0D1), more preferably not larger than 5.0D1 (when 0.6D2≦5.0D1) or not larger than 0.6D2 (when 0.6D2>5.0D1).

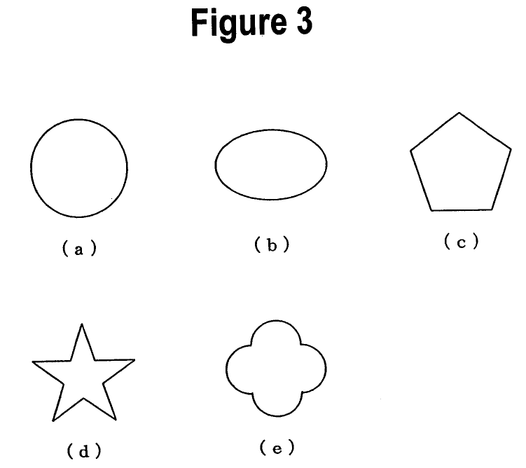

The shape of the cross section of the outlet of the oxidizing agent feed pipe is not limited to a circular shape shown in FIG. 3(a) and may be any shape. For example, it may be oval, polygonal, starry or petaline as shown in FIGS. 3(b) to (e).

From the viewpoint of promoting mixing of the oxidizing agent and the carbon-containing raw material, a polygonal, starry or petaline cross section which can widen the surface area of the potential core of the jet is preferred.

Further, V1 and V2 with respect to a given cross section shape are the flow velocities at the outlets of the oxidizing agent feed pipe and the carbon-containing raw material feed pipe which are estimated in terms of equivalent hydraulic diameter. The equivalent hydraulic diameter (d) is represented by the following:

d=4 m

m=A/P

A: cross sectional area, and P: length of a wetted perimeter.

The internal diameter D3 of the upper part of the reformer and the length L2 of the partial oxidation reaction zone are determined as appropriate according to the amounts of an oxidizing agent and carbon-containing raw material to be processed in consideration of economical efficiency and the like. The length L2 of the partial oxidation reaction zone is associated with D3 and selected from such a length that the temperature of a partial oxidation reaction gas is approximately uniform at the low end surface of the partial oxidation reaction zone (in FIG. 1., a cross section of the vessel at the bottom of the cylindrical portion having an internal diameter of D3). Further, the internal diameter D3 is associated with the internal diameter D4 of the catalyst layer and the length L3 of the conical part, and D3, D4, L2 and L3 may be determined by use of a three dimensional Computational Fluid Dynamics (CFD analysis) technique including a partial oxidation reaction so that the produced partial oxidation reaction gas is fed to the catalyst layer approximately uniformly.

### EXAMPLES

### Example 1

By use of a reformer having the configuration shown in FIG. 1 (the central axis of an oxidizing agent feed pipe and the central axis of a carbon-containing raw material feed pipe form an angle of 90°), 2,000,000 NM³/h of synthesis gas was produced. The unit “NM³” means m³ (cubic meters) at 0° C. and 0.101 MPa.

A carbon-containing raw material comprised steam, hydrogen, carbon monoxide, carbon dioxide and methane as essential components and was supplied at 2 MPa, 800° C. and 1,500,000 NM³/h. As an oxidizing agent, oxygen obtained from an oxygen separator was supplied at 2 MPa, 150° C. and 79,000 NM³/h. The compositions of the carbon-containing raw material and the oxidizing agent are shown in Tables 1 and 2, respectively.

TABLE 1
Conditions for Carbon-Containing Raw Materials

| | | |
|---|---|---|
| | Example 1 | Comp. Example 1 |
| Components | mol % | mol % |

| | | |
|---|---|---|
| Steam | 34.0 | 34.0 |
| Hydrogen | 44.4 | 44.4 |
| Carbon Monoxide | 7.5 | 7.5 |
| Carbon Dioxide | 6.1 | 6.1 |
| Methane | 7.9 | 7.9 |
| Nitrogen | 0.1 | 0.1 |
| Argon | 0.0 | 0.0 |
| Flow Rate (NM³/h) | 1,500,000 | 210,000 |

TABLE 2
Conditions for Oxidizing agents

| | Example 1 | Comp. Example 1 |
|---|---|---|
| Components | mol % | mol % |
| Nitrogen | 0.4 | 0.4 |
| Argon | 1.6 | 1.6 |
| Oxygen | 95.0 | 95.0 |
| Steam | 3.0 | 3.0 |
| Flow Rate (NM³/h) | 79,000 | 11,000 |

The internal diameter D1 of the oxidizing agent feed pipe was 165 mm, the internal diameter D2 of the carbon-containing raw material feed pipe was 1,550 mm, the internal diameter D3 of the upper part of the reformer was 2,500 mm, the internal diameter D4 of the lower part of the reformer was 5,000 mm, the mixing position L1 of the carbon-containing raw material was 850 mm, the length L2 (distance in a flowing direction from the intersection point 40 to the conical part) of the partial oxidation reaction zone was 7,000 mm. Further, the feed flow velocity V1 of the oxidizing agent was 80 m/sec, and the feed flow velocity V2 of the carbon-containing raw material was 43 m/sec. Further, the oxidizing agent feed pipe and the carbon-containing raw material feed pipe had circular cross sections.

As a result of measurements made by thermocouple thermometers disposed at even intervals in the upper part of the reformer, the peak of a high temperature zone generated by a partial oxidation reaction under the conditions was situated about 2,000 mm lower than the tip of the oxidizing agent feed pipe, and the temperature in the vicinity of the outlet of the oxidizing agent feed pipe was almost the same as the temperature of the fed oxidizing agent gas. Further, as a result of observing changes in temperature distribution with time by the above thermocouple thermometers, it was found that the high temperature partial oxidation reaction zone under the above conditions was formed stably. In addition, as a result of observing the temperature distribution of a partial oxidation reaction gas to be fed to the top surface of a catalyst layer by a plurality of thermocouple thermometers placed on the top surface of the catalyst layer, the temperature distribution was within ±4%.

### Comparative Example 1

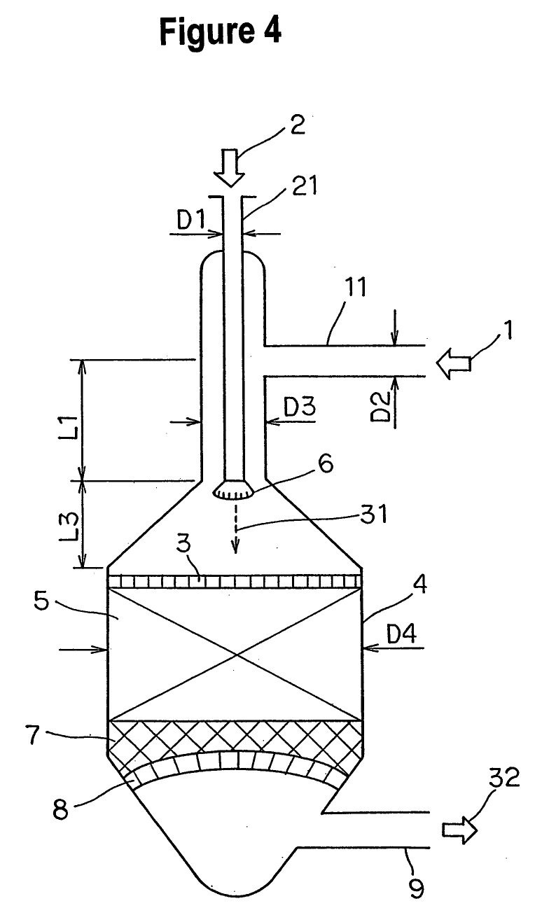

By use of a reformer of the prior art having the configuration shown in FIG. 4, synthesis gas of 280,000 NM³/h was produced. In this example, in contrast to the reformer of the present invention, the central axis of an oxidizing agent feed pipe and the central axis of a carbon-containing raw material feed pipe intersect with each other upstream of the outlet of the oxidizing agent feed pipe in an oxidizing agent flowing direction, and an oxidizing agent and a carbon-containing raw material are fed to a partial oxidation reaction zone parallel to each other.

The carbon-containing raw material comprised steam, hydrogen, carbon monoxide, carbon dioxide and methane as essential components and was supplied at 2 MPa, 800° C. and 210,000 NM³/h. As the oxidizing agent, oxygen obtained from an oxygen separator was supplied at 2 MPa, 150° C. and 11,000 NM³/h. The compositions of the carbon-containing raw material and the oxidizing agent are shown in Tables 1 and 2, respectively.

The internal diameter D1 of the oxidizing agent feed pipe was 130 mm, the internal diameter D3 of the upper part of the reformer was 1,000 mm, the internal diameter D4 of the lower part of the reformer was 5,000 mm, the mixing position L1 of the carbon-containing raw material was 3,000 mm, and the length L3 of a partial oxidation reaction zone (conical part) was 2,000 mm. The average flow velocity of the oxidizing agent at the outlet of the oxidizing agent feed pipe was 18 m/sec, and the average flow velocity of the carbon-containing raw material at the outlet of the carbon-containing raw material feed pipe was 12 m/sec.

Under the conditions, a high temperature zone generated by a partial oxidation reaction was situated about 100 mm lower than the tip of the nozzle of the oxidizing agent feed pipe, and the high temperature zone was generated in the vicinity of the burner nozzle of the oxidizing agent feed pipe. Further, as a result of measurements made by a plurality of thermocouple thermometers placed on the top surface of a catalyst layer, the temperature distribution on the top surface of the catalyst layer varied within a range of ±10%, indicating that a high temperature gas was not fed to the catalyst layer uniformly.

As shown in Comparative Example 1, in the reformer of the prior art, since the high temperature partial oxidation zone is formed right under the burner nozzle of the oxidizing agent feed pipe, the burner nozzle is exposed to high temperatures and may be damaged or corroded by heat.

Meanwhile, as is understood from Example 1, in the reformer of the present invention, since the high temperature partial oxidation reaction zone is formed about 2,000 mm below the outlet of the oxidizing agent feed pipe and is not formed right under the outlet of the oxidizing agent feed pipe, the oxidizing agent feed pipe does not reach high temperatures and can therefore be prevented from being damaged or corroded by heat.

Further, as is understood from Example 1, according to the present invention, a stable partial oxidation reaction due to strong diffusive mixing of jets can be achieved, the temperature of the inlet surface of the catalyst layer becomes approximately uniform, and reaction efficiencies of the reactions in the catalyst layer are kept good.

Numerous modifications and variations of the present invention are possible in light of the above teachings. It is therefore to be understood that within the scope of the appended claims, the invention may be practiced otherwise than as specifically described herein.

What is claimed is:

1. A reformer for obtaining a synthesis gas by partially oxidizing a carbon-containing raw material and then steam-reforming the oxidized raw material,
the reformer comprising:
a reactor vessel,
an oxidizing agent feed pipe for feeding an oxidizing agent into the vessel, and
a carbon-containing raw material feed pipe for feeding the carbon-containing raw material into the vessel,
wherein the central axis of the oxidizing agent feed pipe and the central axis of the carbon-containing raw material feed pipe intersect with each other downstream of the outlet of the oxidizing agent feed pipe in an oxidizing agent flowing direction and downstream of the outlet of the carbon-containing raw material feed pipe in a carbon-containing raw material flowing direction.

2. The reformer according to claim 1, wherein the central axis of the oxidizing agent feed pipe and the central axis of the carbon-containing raw material feed pipe intersect with each other at an angle of 80 to 100°.

3. The reformer according to claim 1, wherein the following relationships are satisfied:
40≦V1≦150;
0.2V1≦V2≦0.8V1; and
min(0.5D2, 7.0D1)≦L1≦max(0.5D2, 7.0D1),
wherein D1 (m) is an equivalent hydraulic diameter of the outlet of the oxidizing agent feed pipe,
D2 (m) is an equivalent hydraulic diameter of the outlet of the carbon-containing raw material feed pipe,
V1 (m/sec) is an average flow velocity of oxidizing agent jet at the outlet of the oxidizing agent feed pipe,
V2 (m/sec) is an average flow velocity of carbon-containing raw material jet at the outlet of the carbon-containing raw material feed pipe, and
L1 (m) is a distance from the outlet-end of the oxidizing agent feed pipe to an intersection point where the central axis of the oxidizing agent feed pipe and the central axis of the carbon-containing raw material feed pipe intersect with each other.

4. The reformer according to claim 1, wherein the cross section of the outlet of the oxidizing agent feed pipe has a circular, oval, polygonal, starry or petal shape.
